(19) United States
(12) Patent Application Publication
(10) Pub. No.: US 2026/0294580 A1
(43) Pub. Date: Oct. 1, 2026

(54) AN ILLUMINATION DEVICE AND ASSOCIATED MEDICAL DEVICE

(71) Applicant: HEGENBERGER MEDICAL LTD, Oxfordshire (GB)

(72) Inventors: Ross Robertson, Oxfordshire (GB); Tim Eagle, Oxfordshire (GB); Malene Hegenberger, Oxfordshire (GB)

(21) Appl. No.: 19/475,780

(22) PCT Filed: Apr. 12, 2024
(86) PCT No.: PCT/GB2024/050968

§ 371 Date: Oct. 16, 2025

(30) Foreign Application Priority Data
Apr. 18, 2024 (GB) 2305687.2

(51) Int. Cl. A61B 90/30; A61B 17/02

(52) U.S. Cl. CPC A61B 90/30; A61B 17/02

(57) ABSTRACT

Described is an illumination device for a medical device, the illumination device includes: a body having an illumination source; a first coupling portion extending from the body, wherein the first coupling portion is shaped such that it can one of: be received and removably retained within a second coupling portion of the medical device; or receive and removably retain the second coupling portion, wherein the first coupling portion includes a depressible switch switchable from a neutral state to a depressed state, wherein the depressible switch is positioned such that, when the first and second coupling portions are coupled together, the depressible switch engages with a surface of the second coupling portion and, thereby, is switched into the depressed state; wherein the illumination source is coupled to the depressible switch such that a change from the neutral state to the depressed state causes the illumination source to provide illumination.

The present disclosure relates to an illumination device for a medical device, the associated medical device, a medical kit comprising the illumination device and the medical device and a method of providing illumination to an examination or suturing site. More specifically, the present disclosure relates to an illumination device which is configured to couple to a medical device, such as a medical retractor, such that illumination can be provided automatically upon coupling the illumination device to the medical device.

### BACKGROUND OF THE INVENTION

A medical device known as a medical retractor is frequently used to dilate or enlarge cavities, tears, or wounds in the human body for medical examination or operation. For applications such as postpartum suturing procedures, it is important for the operator to have a clear view of and access to the vaginal walls in order to effectively perform examination and suturing.

### SUMMARY OF THE INVENTION

According to a first aspect of the present disclosure, there is provided an illumination device for a medical device wherein the illumination device comprises: a body comprising an illumination source; a first coupling portion extending outwards from the body, wherein the first coupling portion is shaped such that it can one of: be received and removably retained within a complementary second coupling portion of the medical device; or receive and removably retain the complementary second coupling portion, wherein the first coupling portion comprises a depressible switch switchable from a neutral state to a depressed state and wherein the depressible switch is positioned on the first coupling portion such that, when the first coupling portion is coupled with the second coupling portion, the depressible switch engages with a surface of the second coupling portion and, thereby, is switched into the depressed state; wherein the illumination source is coupled to the depressible switch such that a change from the neutral state to the depressed state causes the illumination source to provide illumination.

In one or more embodiments, the first coupling portion may be configured to engage with the complementary second coupling portion such that the illumination device can rotate relative to the medical device with at least one rotational degree of freedom when the illumination device is coupled to the medical device.

In one or more embodiments, the first coupling portion may be configured to engage with the complementary second coupling portion such that the illumination device can rotate relative to the medical device with three rotational degrees of freedom when the illumination device is coupled to the medical device.

In one or more embodiments, the first coupling portion may be one of a ball or a socket and the second coupling portion of the medical device is the other of the socket or the ball such that the illumination device is configured to couple to the medical device by way of a ball and socket coupling.

In one or more embodiments, the first coupling portion and the depressible switch may be configured such that, when engaged with the complementary second coupling portion, the depressible switch is in the depressed state regardless of the rotation of the illumination device relative to the medical device.

In one or more embodiments, the depressible switch may be configured such that, upon decoupling of the first coupling portion and the second coupling portion, the depressible switch returns to the neutral position.

In one or more embodiments, the depressible switch may be connected to the illumination source by a pair of conductive contacts in an illumination circuit such that, when the depressible switch is depressed, the two electrical contacts touch so as to complete a circuit and provide power to the illumination source.

In one or more embodiments, the illumination source may extend away from the body.

In one or more embodiments, an illumination shield may be provided that at least partially extends around the illumination source such that it substantially blocks light from extending in at least one direction.

In one or more embodiments, the body may comprise a manipulation tab for use by a user to move the illumination device when the first coupling portion is engaged with the second coupling portion in order to direct the illumination source in different directions.

In one or more embodiments, the body may be configured to receive a battery therewithin and wherein the body comprises electronics for coupling the battery to the illumination source via the depressible switch such that an inserted battery provides required power to turn on the illumination source when the depressible switch is in the depressed state.

According to a second aspect of the present disclosure, there is provided a medical device comprising: a vaginal assembly comprising at least two longitudinal separating elements connected at a distal end for insertion into the vagina of a subject, wherein each longitudinal separating element further extends away from the distal end to a proximal end; and a second coupling portion located at the proximal end of a first of the longitudinal separating elements, wherein the second coupling portion is shaped such that it can one of: receive and removably retain a complementary first coupling portion of an illumination device; or be received and removably retained within a complementary first coupling portion of the illumination device, wherein the medical device is configured to hold the vaginal walls of the subject apart, thereby permitting one or both of examination and suturing of at least the posterior part of the vaginal tissue.

In one or more embodiments, each of the longitudinal separating elements may comprise a second coupling portion located at the proximal end of the longitudinal separating elements, wherein each of the second coupling portions are shaped such that they can one of: receive and removably retain a complementary first coupling portion of an illumination device; or be received and removably retained within a complementary first coupling portion of the illumination device.

In one or more embodiments, the medical device may be a medical retractor.

According to a third aspect of the present disclosure, there is provided a medical kit comprising the medical device of the second aspect and the illumination device of the first aspect.

According to a fourth aspect of the present disclosure, there is provided a method of providing illumination to an examination or suturing site wherein the method comprises: providing a medical kit a according to the third aspect; inserting the distal end of the medical device into the vagina of the subject; coupling the illumination device to the medical device by coupling the first coupling portion to the complementary second coupling portion; and manipulating the illumination device such that illumination is directed towards the examination or suturing site.

### DESCRIPTION OF DRAWINGS

The embodiments of the invention, together with its advantages, may be best understood from the following detailed description taken in conjunction with the accompanying figures.

FIG. 1 shows an example embodiment of an illumination device;

FIG. 2 shows a cross-sectional view of an example embodiment of an illumination device;

FIG. 3 shows perspective view of an example embodiment of a medical device having two illumination device connected thereto; and

FIG. 4 shows an example embodiment of a method for providing illumination to an examination or suturing site.

### DETAILED DESCRIPTION

The present disclosure relates to an illumination device for providing illumination to an examination or suturing site and a medical device configured to have the illumination device coupled thereto. The medical device is designed to hold the vaginal walls of a subject apart and the illumination device, when engaged by a complementary coupling structure having portions located on each of the medical device and the illumination device, is configured to provide illumination to the intended site. In one or more embodiments, the position of the illumination device relative to the medical device may be adjustable such that illumination can be directed as desired by a user. For example, the illumination device may be rotatable with one or more degrees of freedom relative to the medical device. In particular, the illumination device may be configured to provide illumination automatically upon coupling to the medical device irrespective of the rotation of the illumination device relative to the medical device.

FIG. 1 shows an example illumination device according to the present disclosure. The illumination device 101 comprises a body 102 having an illumination source 103. More specifically, the body 102 may comprise an aperture out of which illumination can be shone in embodiments where the illumination source 103 is housed within the body 102. In other examples, the illumination source 103 may be arranged such that it extends away from the body 102, through the aperture. For example, the illumination source 103 may extend out from the body 102 by from about 1 to about 5 mm, such as from about 2 to about 4 mm, for example from about 2.5 to about 3.5 mm or substantially about 3 mm. By extending away from the body 102, diffuse illumination may be provided which illuminates a wider area than embodiments wherein the illumination source 103 is housed within the body 102. Diffuse illumination may be advantageous for properly illuminating the entirety of an examination or suturing site.

In one or more embodiments, the body 102 of the illumination device 101 comprises a first major surface 104 opposite a second major surface 105 wherein the length and width of the major surfaces 104, 105 are each greater than the distance between the first and second major surfaces 104, 105. In this way, the body 102 of the illumination device 101 is a substantially thin body 102. The major surfaces 104, 105 may be substantially circular, ovular, rectangular or another shape. The illumination source 103 may be located at a first end of the body 102. The first end of the body 102 may not be either of the major surfaces 104, 105 and, instead, may be a surface which extends between the two major surfaces 104, 105. In examples where the major surfaces 104, 105 do not have substantially circular or square shapes, the first end may at a shorter edge of the shape as compared to one or more other edges of the shape.

While a body having a first major surface 104 and a second major surface 105 is provided as an example herein which may be particularly easy to manipulate in embodiments where the position of the illumination device 101 is adjustable relative to the position of the medical device, the body 102 may alternatively comprise a shape that does not have such major surfaces 104, 105 separated by a smaller perimeter surface. For example, the body 102 could be a sphere, ovoid or polyhedron.

The illumination source 103 may be any suitable illumination source 103 such as a light emitting diode (LED), halogen, incandescent, compact fluorescent or another type of illumination source 103. The colour of the illumination source 103 will be any colour suitable for illuminating an area for examination or suturing. For example, the illumination source may provide a “cold” light illumination having a colour of from about 3400 to about 6500 Kelvin. Alternatively, the illumination source may provide a warm illumination having a colour of from about 2700 to about 3300 Kelvin. Other colours of illumination source 103 may also be used. It will be appreciated that, herein, the phrase “illumination source” refers to a source of illumination that provides light within the visible spectrum.

The illumination device further comprises a first coupling portion 106 extending outwards from the body 102. The first coupling portion 106 is shaped such that it can either: be received and removably retained within a complementary second coupling portion of the medical device; or receive and removably retain the complementary second coupling portion. That is, the first coupling portion 106 of the illumination device 101 is configured to engage with a second coupling portion of the medical device in order to provide a releasable coupling between the two.

The removable retention of one of the coupling portions within the other coupling portion is provided so that the illumination device 101 can be decoupled from the medical device. This may provide the advantage that the medical device can be put into place for dilating or enlarging the desired cavity, tear or wound and, subsequently, the illumination device 101 can be engaged with the medical device in order to provide illumination as desired. Additionally, providing an illumination device 101 that is removably couplable to the medical device allows for easy replacement of the illumination device in the case of a failure in the electronics, illumination source 103 or another component of the illumination device 101. This may lead to a reduction in waste in the case of failure and, thereby, make the medical kit as a whole more environmentally friendly (the medical kit comprising the medical device and the illumination device).

In some embodiments, the first coupling portion 106 may be configured to engage with the complementary second coupling portion such that the illumination device 101 can rotate relative to the medical device with at least one rotational degree of freedom when the illumination device 101 is coupled to the medical device. In some embodiments, the illumination device 101 is configured to be rotatable about at least a first rotational axis extending between the first coupling portion 106 and the second coupling portion when the first and second coupling portions are coupled together. That is, the first rotational axis may extend from a centroid of the first coupling portion 106 directly through a centroid of the second coupling portion. A second rotational axis and a third rotational axis may be defined wherein the first, second and third rotational axes are each orthogonal to one-another. In embodiments wherein the illumination device 101 is rotatable relative to the medical device with a single rotational degree of freedom, rotation may be about any of the three orthogonal axes. In one or more embodiments, rotation of the illumination device 101 relative to the medical device may be limited about the second and third rotational axes due to the abutment of the illumination device 101 with parts of the medical device or as a result of limitations of structures of the first and second coupling portions.

Providing for rotation about at least one axis of rotation of the illumination device may allow a user to manipulate the illumination source 103 during examination or suturing in order to ensure that the optimal illumination is provided to the site. In some embodiments, the first coupling portion 106 may be configured to engage with the complementary second coupling portion such that the illumination device 101 can rotate relative to the medical device with three rotational degrees of freedom when the illumination device 101 is coupled to the medical device.

The rotational freedom of the first coupling portion 106 relative to the second coupling portion may be provided by way of providing the coupling as a ball-and-socket joint. In some embodiment, the first coupling portion 106 may be the ball and the second coupling portion may be the socket. In other embodiments, the first coupling portion 106 may be the socket and the second coupling portion may be the ball. It will be appreciated that a ball of a ball and socket joint may not be perfectly spherical. The ball may be a sphere, an ovoid, a capsule or another shape which still provides the required rotatable coupling within a socket. In embodiments with a non-spherical ball, the socket would be appropriately shaped to receive and retain a complementary non-spherical ball.

It will further be appreciated that the socket of the ball and socket joint may have a number of different physical structures. The socket may have a solid revolution (such as a cup or egg-cup shape) or may be comprised of a series of resiliently deformable limbs which extend outwards from a surface of the medical device or the illumination device 101 which together provide the socket. In such an embodiment, the resiliently deformable limbs are configured to deform in order to allow the ball to be pushed within a cage of resiliently deformable limbs and to hold the ball in place in such a manner that the ball can rotate within the cage of resiliently deformable limbs. The ball is also removable in such an embodiment by applying a sufficient force to separate the ball and the socket. In such an embodiment, the resiliently deformable limbs will deform in order to allow the ball to remove from the socket if sufficient force is applied.

The first coupling portion 106 further comprises a depressible switch 107 which is switchable from a neutral state to a depressed state. The depressible switch 107 may be any suitable type of switch that causes switching when depressed by an external force, such as a person pressing down on the switch or a depressing force being otherwise applied. The depressible switch 107 is positioned on the first coupling portion 106 such that, when the first coupling portion 106 is coupled with the second coupling portion, the depressible switch 107 engages with a surface of the second coupling portion and, thereby, is switched into the depressed state. That is, the depressing force is applied by way the engagement of the first coupling portion 106 of the illumination device 101 with the second coupling portion of the medical device. The illumination source 101 is coupled to the depressible switch 107 such that a change from the neutral state to the depressed state causes the illumination source 101 to provide illumination.

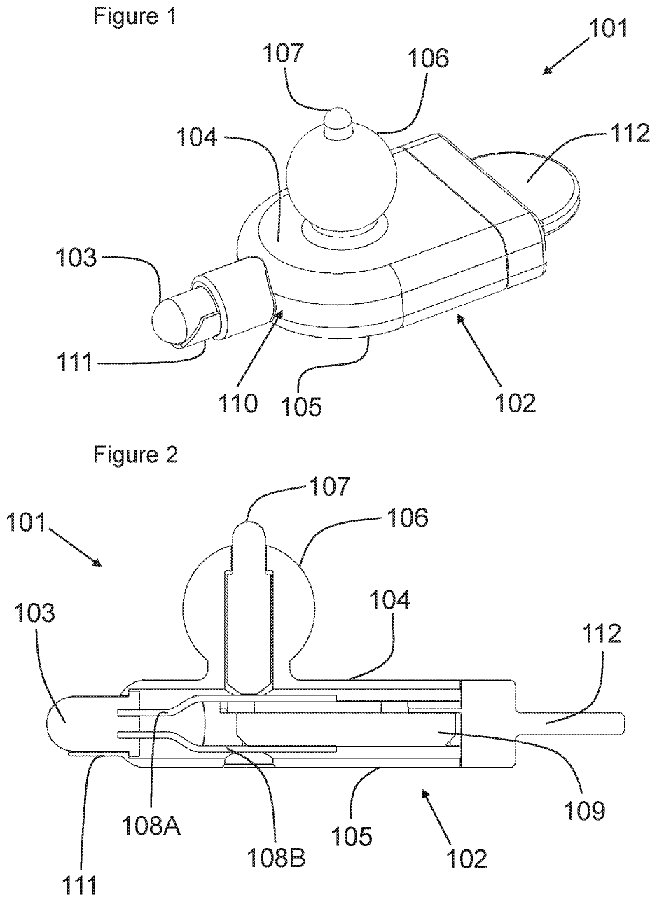

In one or more embodiments, the depressible switch 107 may be a pin which extends partly outwards from the first coupling portion 106 such that it can be moved into the depressed state by the coupling of the first coupling portion 106 with the second coupling portion. In such an embodiment, the depressible switch 107 may extend outwards from the first coupling portion 106 such as the depressible switch 107 shown in FIGS. 1 and 2. In other embodiments, the depressible switch 107 may be a flexible monolithic part of the first coupling portion that is configured to be switchable between: a neutral state where the flexible monolithic part is a convex protrusion from the first coupling portion; and a depressed state where the flexible monolithic part is a concave depression. In such an embodiment, the depressible switch 107 may still be switched from the neutral convex position into the depressed concave position by way of coupling of the first coupling portion 106 with the complementary second coupling portion. In yet other embodiments, the first coupling portion 106 may comprise the flexible monolithic part that can be depressed and a pin-switch or other depressible switch 107 may be provided under the flexible monolithic part such that depression of the flexible monolithic part causes the switching of the depressible switch 107 into the depressed state.

FIG. 2 shows a cross-sectional view of the illumination device 101. The depressible switch 107 may be connected to the illumination source 103 by a pair of conductive contacts 108A, 108B in an illumination circuit such that, when the depressible switch 107 is depressed, the two electrical contacts 108A, 108B touch each other so as to complete a circuit and provide power to the illumination source 103. It will be appreciated that in such an electronic circuit, the two conductive contacts 108A, 108B are connected to a power source such as an external power source or a portable power source such as a battery 109. In one or more alternative embodiments, two or more batteries 109 may be provided in place of the single battery. Alternatively, the depressible switch 107 may be coupled to the illumination source 103 in a different manner that achieves the provision of illumination upon switching of the depressible switch 107 into the depressed state.

In embodiments where the illumination device 101 is rotatable relative to the medical device, the first coupling portion 106 and the depressible switch 107 may be configured such that, when engaged with the complementary second coupling portion, the depressible switch 107 is in the depressed state regardless of the rotation of the illumination device 101 relative to the medical device. In this way, illumination will be provided by the illumination source 103 irrespective of any manipulation of the illumination device 101.

In embodiments wherein the first coupling portion 106 is configured to be received and removably retained within a complementary second coupling portion of the medical device, the second coupling portion may comprise a curved back-profile against which the depressible switch is designed to press such that the depressible switch 107 is maintained in the depressed state when the first coupling portion 106 is coupled to the second coupling portion irrespective of any rotation of the first coupling portion 106. This may be particularly effective in embodiments wherein the first coupling portion 106 is a ball and the second coupling portion is a socket. It will be appreciated that designs may be available where the back-profile of the second coupling portion is not curved but the depressible switch 107 is maintained in the depressed state irrespective of the relative rotation of the illumination device 101 and the medical device, however, the curved back-profile may be particularly beneficial for achieving this reliable depression.

In embodiments wherein the first coupling portion 106 is configured to receive and removably retain the complementary second coupling portion, the depressible switch 107 may be arranged at a central point of the first coupling portion 106 such that when the second coupling portion is retained within the first coupling portion 106, the depressible switch 107 is pushed into the depressed state by the second coupling portion irrespective of any rotation of the first coupling portion 106 relative to the second coupling portion. For example, where the first coupling portion 106 is a socket and the second coupling portion is a ball, the depressible switch 107 may be arranged at a rear surface of the socket such that the ball will always engage with the depressible switch 107 irrespective of the amount that the ball moves within the socket (or, equivalently, the socket moves around the ball).

The depressible switch 107 may be configured such that, upon decoupling of the first coupling portion 106 and the second coupling portion, the depressible switch 107 returns to the neutral position. For example, the depressible switch 107 may be spring loaded such that, upon decoupling of the first coupling portion 106 and the second coupling portion, the depressible switch 107 returns to the neutral position.

In some embodiments, the return of the depressible switch 107 to the neutral state may cause the illumination source 103 to stop providing illumination. This may be particularly beneficial, as it means that if the user couples the illumination device 101 to the medical device while the medical device is in use in an orifice, tear or wound, the user does not need to struggle with turning the illumination device 101 on after coupling and off after decoupling. Such embodiments may also be beneficial from an energy saving and environmental angle as well, as only using power to provide illumination when the illumination device is coupled to the medical device means that energy can be saved overall and batteries will need replacing less often.

In one or more other embodiments, the depressible switch 107 may be configured to latch the illumination source 103 on once the depressible switch 107 has been depressed. The illumination device 101 may further comprise a release mechanism, which may be another switch or may be a different mechanism such as a timer, which is configured to release the latch and turn off the illumination source 103. The release mechanism may be configured such that the latch cannot be released while the first coupling portion 106 and the second coupling portion are coupled together. In this way, illumination is provided for, and can either be selectively deactivated, by way of a separate latch-release mechanism or may turn off after a predetermined amount of time by way of a timer. A latch may be applied either mechanically to latch the depressible switch 107 in the depressed state, or electronically to maintain the illumination until a release mechanism is triggered regardless of the position of the depressible switch.

The first coupling portion 106 may extend outwards from one of the first major surface 104 and the second major surface 105. In the embodiments shown in FIGS. 1 and 2, the first coupling portion 106 extends outwards from the first major surface 104. That is, in some embodiments, the first coupling portion 106 may extend orthogonally from the body 102 relative to the illumination source 103 such that the illumination source 103 extends from or is set in a minor surface 110 of the body 102 wherein a minor surface 110 extends between and connects the first major surface 104 and the second major surface 105.

In some embodiments, the illumination source 103 may be configured to provide illumination in a direction substantially orthogonal to the direction in which the first coupling portion 106 extends from the body of the illumination device 101. It will be appreciated that a non-attenuated or otherwise unblocked illumination source 103 may provide illumination in substantially all directions, however, the positioning of the illumination source 103 and any other attenuating or blocking features of the illumination device 101 may be arranged such that illumination is directed at least in a direction substantially orthogonal to the direction in which the first coupling portion 106 extends from the body 102. For example, in embodiments where the illumination source 103 is contained within the body 102, the aperture may be arranged such that illumination is provided in a direction substantially orthogonal from the direction in which the first coupling portion 106 extends from the body 102. In embodiments wherein the illumination source 103 is configured to extend outwards from the body 102 (such as through an aperture in the body 102), the illumination source 103 may extend outwards from the body 102 in a direction substantially orthogonal to the direction in which the first coupling portion 106 extends from the body 102.

While it is not essential that illumination is provided in a direction substantially orthogonal to the direction in which the first coupling portion 106 extends, this may provide for a particularly simple illumination device 101 which is easy to physically manipulate to illuminated a desired area.

The body 102 may further comprise an illumination shield 111 that is positioned such that it extends at least partially around the illumination source 103 such that it substantially blocks light from extending in at least one direction. One can define a first major side which corresponds to the side of the illumination device 101 on which the first major surface 104 of the body 102 is located and a second major side which corresponds to the side of the illumination device 101 on which the second major surface 105 is located. In embodiments where the first coupling portion 106 extends from the first major surface 104 of the body of the illumination device 101, the illumination shield 111 may be arranged such that it extends at least partially around the illumination source 103 on the second major side such that illumination is inhibited from extending in that direction. This may be particularly advantageous for allowing the examination or suturing site to be illuminated while preventing illumination from shining directly into the eyes of a user.

The body 102 of the illumination device 101 may comprise a manipulation tab 112 for use by a user to move the illumination device 101 when the first coupling portion 106 is engaged with the second coupling portion in order to direct the illumination source 103 in different directions. The manipulation tab 112 may have any suitable shape or configuration that allows it to be easily manipulated by a user. In embodiments where the illumination device 101 is rotatable when the first coupling portion 106 is engaged with the second coupling portion, the manipulation tab 112 may be particularly useful for allowing the user to manipulate the rotation of the illumination device 101. The manipulation tab 112 may be sized such that it is suitable for adjustment by only a single thumb or finger of a user. The manipulation tab 112 may also comprise a ribbed structure or may otherwise be designed so as to improve the grip of a user. For example, the material of the manipulation tab may be selected to improve the grip of a user.

The illumination device 101 may be configured to receive one or more batteries 109 therewithin. The body 102 may comprise electronics for coupling the one or more batteries 109 to the illumination source 103 via the depressible switch 107 such that the one or more inserted batteries 109 provide required power to turn on the illumination source when the depressible switch 107 is in the depressed states. The required electronics to provide power from the one or more batteries 109 to the illumination source 103 may simply comprise conductive wires, strips or cables coupled to the illumination source 103 via the depressible switch 107. Alternatively, more complex electronics may be used. The manipulation tab 112 may further be configured to detachably removable from the remainder of the body 102. The removal of the manipulation tab 112 may provide access to a battery socket for one or more batteries 109 to be inserted. The additional grip provided by the manipulation tab 112 for physical manipulation may, thereby, provide the additional benefit of making the manipulation tab 112 easier to remove for providing access to the battery socket.

As used herein, when the medical device referred to as being designed to hold the vaginal walls of a subject apart this is referred to as a retractor. To recap, the term “retractor” is used herein to define a device that may be used to dilate or enlarge cavities, tears, or wounds in the human body for medical examination or operation. In the context of the present invention, the term “retractor” encompasses the use of such a device as a “speculum”. A speculum is the term typically used to refer to devices that are used to dilate or enlarge natural cavities/orifices, and the skilled person will understand that a device that is referred to as a “retractor” may also be used as a “retractor”.

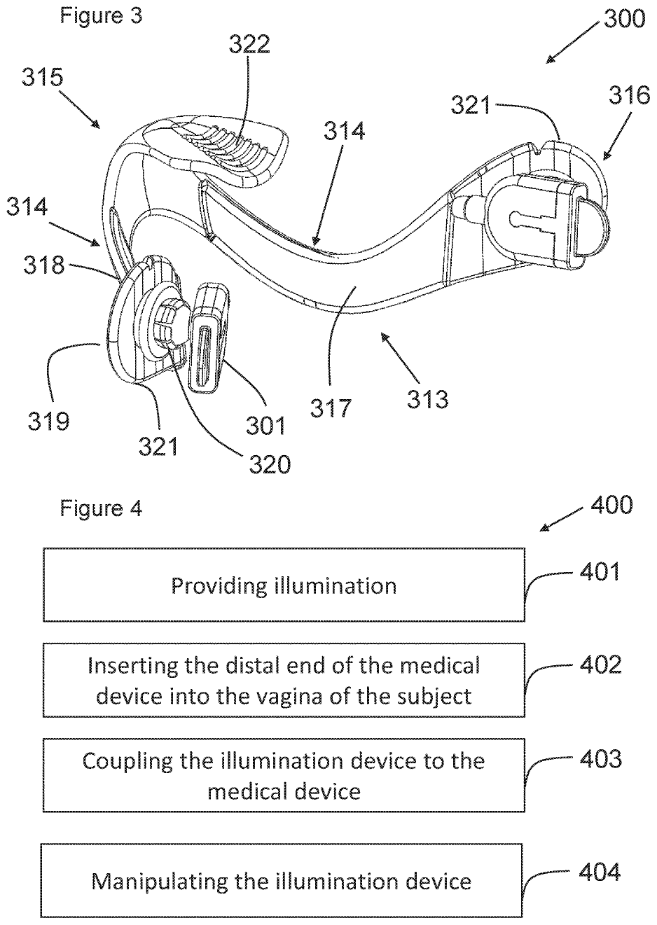

FIG. 3 shows an example embodiment of a medical kit 300 comprising a medical device 313 coupled to two illumination devices 301. The medical device 313 may be a vaginal retractor that permits examination and/or suturing of at least the posterior part of the vagina. While a vaginal retractor 313 is provided as example herein, the medical device 313 may be a vaginal speculum. The vaginal retractor 313 may be designed to provide a more clear and less-obstructed view of at least the posterior part of the vagina compared to other solutions. In one or more embodiments, the medical device 313 may be configured to exert sufficient force on the vaginal walls for holding the walls apart and exposing at least the posterior part of the vagina. The medical device 313 may comprise a locking element configured for engagement with the medical device 313 for creating a transverse separation force at a proximal end of separating elements 314 of the medical device 313 for holding the vaginal walls of the subject apart. The locking element may be an integral part of the medical device 313 or it may be configured for detachable engagement with the medical device 313.

The medical device 313 comprises a vaginal assembly and the vaginal assembly comprises at least two longitudinal separating elements 314 connected at a distal end 315 for insertion into the vagina of a subject. The separating elements 314 may be configured to extend divergently away from the distal end 315 such that, at rest, the angle between the first and the second separating elements 314 is an acute angle. The separating elements 314 may extend divergently away from the distal end 315 such that they end at a proximal end 316. The terms “distal end” 315 and “proximal end” 316 are provided here for convenience and refer to the ends being either proximal or distal to a user when used under normal conditions, however, it will be appreciated that no particular special meaning is implied by the use of “proximal” and “distal” other than to provide an indication that these ends are opposing and that they may have a particular placement relative to a user during typical use.

A first surface of each separating element 314 can be defined as an inner surface 317 and a second opposing surface of each separating element can be defined as an outer surface 318. The inner surface 317 of each separating element 314 substantially faces towards the inner surface 317 of the other separating element 314. The outer surface 318 of each separating element 314 substantially faces away from the other separating element 314 on an opposing side of the separating element 314. The separating elements 314 may be curved such that the angle between the two separating elements 314 increases as they extend proximally away from the distal end 315. The proximal end 316 of the outer surface 318 of each separating element 314 may comprise a grip pad 319 wherein the grip pad 319 is configured to be gripped by one or more fingers or thumbs of a user. The medical device 313 may be sized such that a user can grip each of the two grip pads 319 with one or more fingers or a thumb of a single hand so that the user can press the proximal ends of the medical device 313 together. Pressing the proximal ends 316 of the elongate separating elements 314 together via the grip pads 319 may cause the medical device 313 to deform such that it is easier to insert into a desired location. Upon releasing the proximal ends 316 of the elongate separating elements 314, the medical device 313 may be configured to return to its at-rest shape. The grip pads 319 may also comprise a ribbed structure or may otherwise be designed so as to improve the grip of a user. For example, the material of the grip pads 319 may be selected to improve the grip of a user.

The second coupling portion 320 is located at the proximal end 316 of at least one of the longitudinal separating elements 314. As provided for above, the second separating element 320 is configured to one of: receive and removably retain a complementary first coupling portion 106 of an illumination device 301; or be received and removably retained within a complementary first coupling portion 106 of the illumination device 101. More specifically, the second coupling portion 320 may be positioned on the inner surface 317 of one of the elongate separating members 314. The second coupling portion 320 may, for example, be arranged such that it is directly opposite the grip pad 319 of one of the elongate separating members 314. In one or more embodiments, each longitudinal separating element 314 comprises a second coupling portion 320 located at the proximal end 316 of the respective longitudinal separating elements 314, wherein each of the second coupling portions 320 are configured to receive and removably retain a complementary first coupling portion 106 of an illuminator device 101; or be received and removably retained within a complementary first coupling portion 106 of the illumination device 301. Providing two illumination devices 301 arranged symmetrically around the wound, by way of their identical placement on the symmetrically shaped and arranged separating elements 314, may be particularly advantageous, as it may allow for the provision of illumination to the target site in the case that operating implements or parts of the user block one of the illumination sources 103 during use.

The proximal ends 316 of the elongate members 314 at which the grip pads 319 and second coupling portions 320 are located may be a proximal base element 321 which is configured to rest on the skin outside the vagina after the medical assembly has been inserted inside the vagina. The proximal base elements 321 may be flattened and curve outwards from the separating elements 314 such that they can rest on the skin outside the vagina after the medical device 313 has been inserted into the vagina. The proximal base elements 321 may be flattened and extend transversely such that they can rest on the skin outside the vagina after the medical device 3131 has been inserted into the vagina. One purpose of the proximal base elements 321 is that they may provide better comfort for the subject when the medical device is inserted into the vagina and to provide better protection of the sensitive tissue of the subject when in contact with the medical device 313. Furthermore, the proximal base elements 321 are preferably configured for resting against, separating, and holding apart the labia majora and labia minora.

In one or more embodiments, the medical device 313 is shaped such that at least the posterior part of the vaginal tissue is exposed for examination and/or suturing when the medical device 313 is inserted into the vagina and the locking element is engaged with the medical device 313.

In some embodiments the medical device 313 is furthermore configured for supporting the anterior vaginal wall after insertion into the vagina. Part of the elongate separating member 314 may comprise an anterior vaginal wall segment 322 configured for supporting the anterior vaginal wall. In one embodiment the anterior vaginal wall segment 322 is connected at the distal end 315 of the medical device 313, extending in the proximal direction from the distal end 315, and preferably in the shape of a tongue such as a flattened and rounded shape. The anterior vaginal wall segment 322 may be made from an elastic material such that it is configured to support the anterior vaginal wall. The anterior vaginal wall segment 322 may thereby at least temporarily deform when inserted into the vagina such that it exerts a force on the anterior vaginal wall of the subject. In another embodiment the medical device 313 is configured for supporting the pressure exerted by the bladder on the anterior vaginal wall of the subject.

It will be appreciated that the illumination device 301 may be provided to a user in isolation from the medical device 313 or visa versa. For example, the illumination device 301 may be provided as a replacement part for a lost or damaged illumination device 3 301. As such, the illumination device 301 and the medical device 313 are complimentary products designed to co-operate but capable of being provided independently.

FIG. 4 shows an example method of providing illumination to an examination or suturing site according to the present disclosure. The method comprises providing a medical kit, wherein the medical kit comprises a medical device, such as a vaginal retractor, and at least one illumination device. The method further comprises inserting the distal end of the medical device into the vagina of the subject. The proximal ends of the elongate separating elements of the medical device may be pressed together in order to deform the medical device into a shape that is suitable for insertion prior to insertion into the vagina of the subject. Upon releasing the proximal ends, the medical device may return to an at-rest state which provides the necessary support. The method further comprises coupling the illumination device to the medical device by coupling the first coupling portion of the illumination device to the complementary second coupling portion of the medical device. It will be appreciated that this step may be performed after insertion of the medical device into the vagina of the subject, however, the coupling may equally be performed before insertion of the medical device into the vagina. No limitation is implied by the location of this method step in the method. The method may then further comprise, as needed, manipulating the illumination device such that illumination is directed towards the examination or suturing site. It will be appreciated that the degree of manipulation required may be extremely small if the illumination device is coupled to the medical device at a good angle, or significant manipulation (rotation about between one and three rotational axes in between one and three degrees of rotational freedom) if the illumination device is engaged at an angle that does not direct the illumination in the desired direction.

1. An illumination device for a medical device wherein the illumination device comprises:
a body comprising an illumination source;
a first coupling portion extending outwards from the body, wherein the first coupling portion is shaped such that it can one of:
be received and removably retained within a complementary second coupling portion of the medical device; or
receive and removably retain the complementary second coupling portion,
wherein the first coupling portion comprises a depressible switch switchable from a neutral state to a depressed state and wherein the depressible switch is positioned on the first coupling portion such that, when the first coupling portion is coupled with the second coupling portion, the depressible switch engages with a surface of the second coupling portion and, thereby, is switched into the depressed state;
wherein the illumination source is coupled to the depressible switch such that a change from the neutral state to the depressed state causes the illumination source to provide illumination.

2. The illumination device of claim 1 wherein the first coupling portion is configured to engage with the complementary second coupling portion such that the illumination device can rotate relative to the medical device with at least one rotational degree of freedom when the illumination device is coupled to the medical device.

3. The illumination device of claim 2, wherein the first coupling portion is configured to engage with the complementary second coupling portion such that the illumination device can rotate relative to the medical device with three rotational degrees of freedom when the illumination device is coupled to the medical device.

4. The illumination device of claim 3, wherein the first coupling portion is one of a ball or a socket and the second coupling portion of the medical device is the other of the socket or the ball such that the illumination device is configured to couple to the medical device by way of a ball and socket coupling.

5. The illumination device of claim 2, wherein the first coupling portion and the depressible switch are configured such that, when engaged with the complementary second coupling portion, the depressible switch is in the depressed state regardless of the rotation of the illumination device relative to the medical device.

6. The illumination device of claim 1, wherein the depressible switch is configured such that, upon decoupling of the first coupling portion and the second coupling portion, the depressible switch returns to the neutral position.

7. The illumination device of claim 1, wherein the depressible switch is connected to the illumination source by a pair of conductive contacts in an illumination circuit such that, when the depressible switch is depressed, the two electrical contacts touch so as to complete a circuit and provide power to the illumination source.

8. The illumination device of claim 1, wherein the illumination source extends away from the body.

9. The illumination device of claim 7, wherein an illumination shield is provided that at least partially extends around the illumination source such that it substantially blocks light from extending in at least one direction.

10. The illumination device of claim 1, wherein the body comprises a manipulation tab for use by a user to move the illumination device when the first coupling portion is engaged with the second coupling portion in order to direct the illumination source in different directions.

11. The illumination device of claim 1, wherein the body is configured to receive a battery therewithin and wherein the body comprises electronics for coupling the battery to the illumination source via the depressible switch such that an inserted battery provides required power to turn on the illumination source when the depressible switch is in the depressed state.

12. A medical device comprising:
a vaginal assembly comprising at least two longitudinal separating elements connected at a distal end for insertion into the vagina of a subject, wherein each longitudinal separating element further extends away from the distal end to a proximal end; and
a second coupling portion located at the proximal end of a first of the longitudinal separating elements, wherein the second coupling portion is shaped such that it can one of:
receive and removably retain a complementary first coupling portion of an illumination device; or
be received and removably retained within a complementary first coupling portion of the illumination device,
wherein the medical device is configured to hold the vaginal walls of the subject apart, thereby permitting one or both of examination and suturing of at least the posterior part of the vaginal tissue.

13. The medical device of claim 12, wherein each of the longitudinal separating elements comprises a second coupling portion located at the proximal end of the longitudinal separating elements, wherein each of the second coupling portions are shaped such that they can one of:
receive and removably retain a complementary first coupling portion of an illumination device; or
be received and removably retained within a complementary first coupling portion of the illumination device.

14. The medical device of either of claims 12-13 claim 12 wherein the medical device is a medical retractor.

15. A medical kit comprising the illumination device of claim 1.

16. A method of providing illumination to an examination or suturing site wherein the method comprises:
providing a medical kit a according to claim 15;
inserting the distal end of the medical device into the vagina of the subject;
coupling the illumination device to the medical device by coupling the first coupling portion to the complementary second coupling portion; and
manipulating the illumination device such that illumination is directed towards the examination or suturing site.
